(19) United States
(12) Patent Application Publication
(10) Pub. No.: US 2024/0166656 A1
(43) Pub. Date: May 23, 2024

(54) METHOD FOR PREPARING AMIDINES

(71) Applicant: VERSALIS S.P.A., SAN DONATO MILANESE (MI) (IT)

(72) Inventors: Nicola VECCHINI, Mantova (MN) (IT); Federico MONDINI, Mantova (MN) (IT)

(21) Appl. No.: 18/549,412

(22) PCT Filed: Mar. 3, 2022
(86) PCT No.: PCT/IB2022/051866

§ 371 Date: Sep. 7, 2023

(30) Foreign Application Priority Data
Mar. 8, 2021 (IT) 102021000005336

(51) Int. Cl. C07D 487/04; B01J 21/04

(52) U.S. Cl. CPC C07D 487/04; B01J 21/04

(57) ABSTRACT

A method for preparing amidines or derivatives thereof includes the step of subjecting N-(amino-alkyl) lactams to dehydration in the presence of a heterogeneous catalyst selected from a Lewis acid or an acid resin. The process includes selecting an acid catalyst from heterogeneous acid catalysts selected from Lewis acids or Lewis acids with Bronsted acid components, or heterogeneous acid catalysts based on acid resins.

### CROSS-REFERENCE TO RELATED APPLICATIONS

This application is a 35 U.S.C. § 371 National Stage patent application of PCT/IB2022/051866, filed on 3 Mar. 2022, which claims the benefit of Italian patent application 102021000005336, filed on 8 Mar. 2021, the disclosures of which are incorporated herein by reference in their entirety.

### TECHNICAL FIELD

The present disclosure relates to a process for preparing amidines. More specifically, the present disclosure relates to a method for producing amidines such as, for example, 1,8-Diazabicyclo[5.4.0]undec-7-ene (from this point of the document onwards indicated in the abbreviated form DBU), or derivatives thereof starting from lactams, such as ε-caprolactam and α,β unsaturated nitriles, such as for example acrylonitrile.

### BACKGROUND

It is known that DBU is a versatile molecule that lends itself to numerous applications; in fact the chemical reactions in which it can take part are varied. A recent article by Jacques Muzart, “DBU: A Reaction Product Component” Chemistry Select 2020, vol. 5, 11608-11620, gives a detailed summary; they range from the formation of addition salts on C—C double bonds and much more. For these aspects DBU is used in the catalysis of polyurethanes, in the pharmaceutical industry, in ionic liquids and in general in organic syntheses. Further details on the application of the DBU are also described in “1,8-Diazabicyclo [5.4.0] undec-7-ene (DBU): A Versatile Reagent in Organic Synthesis” Bhaskara Nand et al. Current Organic Chemistry, 2015, 19, 790-812.”

In the known art, the industrial production of DBU mainly occurs through three reaction steps. In the first step ε-caprolactam is reacted with acrylonitrile to obtain N-(2-cyanoethyl)-ε-caprolactam. In the second step, N-(2-cyanoethyl)-ε-caprolactam is hydrogenated to the corresponding amine in the presence of anhydrous ammonia and Nickel Raney catalyst. In the third step the N-(3-aminopropyl)-ε-caprolactam is dehydrated by acid catalysis obtaining DBU. The industrially most complex step of the synthesis is represented by hydrogenation in the presence of ammonia. The catalyst normally used is Nickel-Raney which in its activated form is pyrophoric. Anhydrous ammonia is also a toxic gas and requires specific precautions and authorizations for its storage, use and transport.

The patent DE1545855 describes, in the German version and in its English version in the countries of extension, a process for obtaining amidines (limited to the third step of the industrial process previously described) with the following structure:

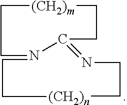

where m is an integer from 3 to 7, and n is an integer from 2 to 4, starting from N-(amino-alkyl) lactams of formula:

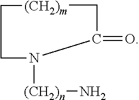

The process takes place through the dehydration of amino lactam catalyzed by mineral or sulphonic acids (e.g. p-toluenesulfonic acid) in the presence of a solvent, e.g. Xylene. The reaction mixture is heated to boiling, the dehydration water that forms is condensed together with the solvent and then separated; the solvent is refluxed into the reaction flask. The patent does not describe the steps preceding dehydration, but refers to the known art.

The patent EP0347757 A2 describes a method for the synthesis of cyanoalkyl lactams (first step of the industrial process previously described) through the reaction of a lactam and α,β unsaturated nitrile using the same DBU as basic catalyst; DBU can also be used as a solvent. The document does not mention the other reaction steps (second and third), but merely refers to the catalytic hydrogenation of the cyano alkyl lactam as per known art; in fact in example 2 the hydrogenation in the presence of Ni Raney and ammonia is described. The patent justifies the use of DBU as a catalyst, replacing KOH used in the first step of the industrial process since, in order to pass from the first step to the second one, it is first necessary to neutralize the base; this is not necessary if the DBU is used (example 3).

Patent CN101279973 B describes a method for the preparation of 1,8-Diazabicyclo-[5.4.0]-undec-7-ene, starting from ε-caprolactam and acrylonitrile, in the presence of tert-butyl or tert-amyl alcohol, as a solvent, and NaOH as a catalyst. The reaction product of this first step is subjected to hydrogenation in the presence of anhydrous ammonia and Ni Raney as catalyst. After the hydrogenation, the mixture is neutralized with sulfuric acid, the solvent is recovered and the reaction product is subjected to dehydration with water removal, as described in the German patent DE1545855.

Patent publication CN109796458 A describes a method for the preparation of 1,8-Diazabicyclo-[5.4.0]-undec-7-ene, always starting from ε-caprolactam and acrylonitrile. This time the document no longer describes the hydrogenation step in the presence of ammonia, but introduces an alternative method using hydroquinone, gaseous anhydrous hydrochloric acid, dichloromethane, sodium perborate and ethylenediaminetetraacetic acid (EDTA). The process is considerably complex with respect to the others described; furthermore, in the face of the elimination of ammonia and Ni-Raney, there is the introduction of a highly aggressive agent (anhydrous HCl) as well as numerous chemical substances.

In patent publication JP2003286257, the first and third steps are carried out in the same way as previously described (reaction of caprolactam with acrylonitrile with basic catalysis by KOH; dehydration by acid catalysis). The second step is instead conducted in the absence of ammonia and with the use of Cobalt Raney as a catalyst. The result is to obtain 86% by weight of reduced product (primary amine of interest).

The patent EP0913388 B1 describes a method for obtaining amines by hydrogenation of nitriles without using ammonia. The novelty lies in the treatment to which the catalyst is subjected. The catalyst (Cobalt Raney or sponge catalyst) is treated with an aqueous solution of lithium hydroxide or, alternatively, the reaction is carried out in the presence of said solution. The catalyst, through this treatment, must incorporate from 0.1 to 100 mmol of lithium hydroxide per gram.

Patent EP0662476 B1 describes the synthesis of bicyclic amidines by reaction of lactones with diamines catalyzed by an acid. The process is carried out in a single reaction step and is followed by purification. The patent also claims the use of these amidines as catalysts for polyurethanes. The synthesis of the DBU is described in example 6 and shows a very low yield of the product, equal to 21%.

Patent publication CN1262274 A describes a method for the preparation of 1,8-diazabicyclo-[5.4.0]-undec-7-ene always starting from ε-caprolactam and acrylonitrile; the peculiarity lies in the use of a mixture of inorganic and organic bases as catalysts in the first reaction step (KOH and DBU). The cyano-derivative obtained is subjected to purification before being subjected to reduction. The second hydrogenation step is carried out in the presence of activated Ni (the catalytic form is not defined) as a catalyst but is not mentioned if in the presence or absence of ammonia. Dehydration is always carried out in acidic conditions, through the use of p-toluenesulphonic acid, and in the absence of solvent; the reaction is continued for a rather long period, i.e. between 35 and 40 hours, obtaining a yield, for this step, of 74.61%.

According to the known art, the final cyclization step to give bicyclic amidine is carried out in an acid catalyst, specifically p-toluenesulfonic acid, which is dissolved in the reaction environment and which requires the use of a high-boiling solvent for conducting the reaction. This translates into a greater complexity of the production plant and of the process—also in relation to the need to separate the catalyst from the reaction mixture—and in a consequent increase in costs.

### SUMMARY

The present disclosure therefore provides an innovative process for the synthesis of amidines which allows a simplification and a reduction of plant, process and maintenance costs.

This purpose is achieved through the use of a suitable heterogeneous catalysis in the dehydration/cyclization reaction, with consequent elimination of both the solvent reflux and the neutralization phase of the mixture (necessary if homogeneous catalysis is used, for example with p-toluenesulfonic acid as catalyst).

In particular, an aim of the present disclosure is the preparation of 1,8-Diazabicyclo-[5.4.0]-undec-7-ene (DBU) (usable in the applications previously described) starting from the corresponding N-(amino-alkyl) lactam, through the use of heterogeneous catalysis and consequent elimination of solvent reflux and neutralization.

The Applicant therefore posed the problem of finding a process for producing amidines starting from N-(amino-alkyl) lactams.

The Applicant has now found a method for the preparation of amidines starting from N-(amino-alkyl) lactams, comprising the dehydration/cyclization of the amino compound to obtain the amidine, which can finally be subjected to a final step of separation and purification to obtain the product in the form suitable for industrial use. This method can be carried out in batch or continuously; continuous mode is preferred.

N-(amino-alkyl) lactams can be prepared using one of the processes described in the state of the art, such as those previously described, or, more preferably, according to the method described in the co-pending Italian patent application entitled “METHOD FOR PREPARING AMIDINE” of the same Applicant, filed on the same day of this application with the number 102021000005321.

The reduction of the nitrile compound, which is the most critical step in the synthesis of N-(amino-alkyl) lactams, is a reaction already known and reported in the literature and widely used in organic synthesis (see, for example, Peter Vollhardt, Chimica Organica pag. 825-826 I Edizione). In the patents listed above it is carried out by means of Raney catalysts (Ni or Co) or in any case in a sponge form, and in the presence of anhydrous ammonia.

Suitable reduction catalysts are commercial or synthetic hydrogenation systems, based on one or more metals of groups 8, 9 and 10 of the periodic table, such as for example Iron, Cobalt, Nickel, or noble metals such as Ruthenium, Rhodium, Palladium, Osmium, Iridium or Platinum. Cobalt, Nickel, Palladium and Platinum are preferred. Cobalt and Nickel are particularly preferred. These catalysts can be used in dispersed, colloidal or supported/bound phase, preferably in supported/bound form on inorganic phase with a high surface area, even more preferably in supported/bound phase on silica, alumina or silica-alumina.

Surprisingly, the Applicant has found that it is possible to conduct the synthesis of amidines with reactions in series, eliminating the solvent before the cyclization/dehydration phase and carrying out a single final purification step without the process presenting criticalities, or requiring separation steps of the intermediates of the desired product from the other reaction products, to ensure an acceptable final purity of the desired product and a high yield and conversion into the desired product in each of the intermediate steps. This aspect therefore makes it possible to simplify the number of devices to be used and to considerably reduce the complexity of the overall process. Optionally, however, it is possible to consider the use of intermediate purification steps if it is appropriate to obtain semi-finished products and/or chemical intermediates of high purity.

These and other advantages are surprisingly achieved by means of the preparation process according to the present disclosure.

Therefore, the present disclosure provides a process for preparing amidines or derivatives thereof of formula (V)

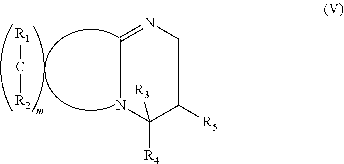

starting from N-(amino-alkyl) lactam having the following formula (IV)

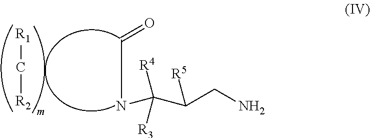

wherein:
R1 is H or an aliphatic hydrocarbon group, optionally substituted, having from 1 to 5, preferably from 1 to 2 carbon atoms, and is more preferably H;
R2 is H or an aliphatic hydrocarbon group, optionally substituted, having from 1 to 5, preferably from 1 to 2 carbon atoms, and is more preferably H;
R3 is H or an aliphatic hydrocarbon group, optionally substituted, having from 1 to 5, preferably from 1 to 2 carbon atoms, and is more preferably H;
R4 is H or an aliphatic hydrocarbon group, optionally substituted, having from 1 to 5, preferably from 1 to 2 carbon atoms, and is more preferably H;
R5 is H or an aliphatic hydrocarbon group, optionally substituted, having from 1 to 5, preferably from 1 to 2 carbon atoms, and is more preferably H;
m is an integer from 3 to 7, more preferably 3 to 6,
wherein even more preferably, (V) is 1,8-Diazabicyclo[5.4.0]undec-7-ene, (DBU),
said process comprising the following step:
   subjecting said amine of formula (IV) to dehydration, with a catalyst based on alumina, silico-aluminas or zeolites, obtaining the corresponding amidine of formula (V). The amidine of formula (V), synthesized as described above according to the present disclosure, can be subjected to subsequent purification with the methods known to those skilled in the art.

According to the present disclosure, the term amidine means a compound derivable from an amide by replacing the oxygen of the carbonyl group ═CO with an imide group ═N—. Preferably, according to the present disclosure, cyclic amidines as defined in formula (V) are also intended.

According to the present disclosure, the term “derivative of amidine” means any compound obtainable from amidine by reaction with a carboxylic acid, epoxy ketone, cloroformates or diesters of carbonic acid.

According to the present disclosure, with the indefinite singular articles a, an, the meaning of at least one is also intended, unless otherwise specified.

### DETAILED DESCRIPTION

According to step (A) of the process according to the present disclosure, a controlled catalytic addition reaction is carried out to obtain the compound of formula (III) with high yields starting from a lactam of formula (I), preferably ε-caprolactam, and a α,β unsaturated nitrile of formula (II), preferably acrylonitrile, in the presence of a suitable basic catalyst.

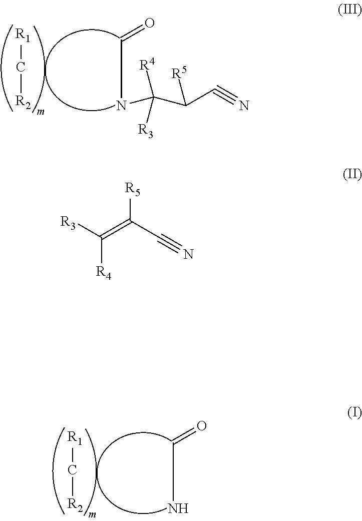

wherein R1, R2, R3, R4, R5 are the same as above defined for formulas (IV) and (V).

In this document, unless otherwise indicated, the percentages are to be understood as percentages in mass.

The amino derivative of lactam of formula (IV), according to the present disclosure, it is subjected to dehydration to give the corresponding amidine, in the preferred case DBU (1,8-Diazabicyclo[5.4.0]undec-7-ene), as described below.

The reaction mixture, coming from the step of hydrogenation, is preferably subjected to recovery of the solvent by evaporation and subsequently to dehydration. Alternatively, even if in a less preferable form of embodiment, the derivative amino of the lactam of formula (IV) can be reacted in purified form.

The dehydration is carried out hot, preferably between 90 and 270° C., more preferably between 130 and 230° C., even more preferably between 150 and 200° C., continuously removing the water produced during the dehydration which operates the cyclization.

The catalyst is always needed and can be selected from heterogeneous acid catalysts selected from Lewis acids or Lewis acids with Bronsted acid components, such as aluminum oxide (γ-Al₂O₃), silico-aluminas (SiO₂—Al₂O₃), earth acids such as lanthanum oxide and zirconium oxide, or heterogeneous resin-based catalysts, such as sulfonated resins or ion exchange resins. Said catalysts can possibly be supported on inert carriers such as, for example, pumice, graphite or silica. Aluminum oxide (γ-Al₂O₃), is preferred. At the end of the reaction, the main product of dehydration is the amidine of interest of formula (V).

If required by end users, amidine can be purified through one of the methods already known in the art, for example by distillation obtaining a purity ranging from 95 to 98% by weight.

Preferably, in the process of the present disclosure no intermediate purifications of the reaction mixture obtained from hydrogenation are carried out, but only possible solvent evaporations for the recovery and use of the same.

The Applicant has therefore surprisingly identified the possibility of carrying out dehydration in the absence of solvent on a solid acid catalyst without refluxing the solvent, in order to facilitate the removal of water, with further simplification of the process and reduction of costs. However, the use of a solvent typically selected from those usable in known hydrogenation reactions to give the intermediate of formula (IV), for example a xylene, is not excluded.

In the present process, the reaction step and the final purification step can be carried out continuously.

In a particularly preferred embodiment of the present disclosure, the Applicant has found a new and original process for the producing amidines from lactams.

All conversion, selectivity and yield values mentioned refer to those determined by ¹H and ¹³C NMR and GC-MS analysis of the reaction mixtures as described in the examples.

The reactant stream coming from the hydrogenation phase can be sent to a solvent recovery system. The preferred setup is that based on an evaporator for the recovery of the solvent. The reaction mixture with traces of solvent comes out from the bottom of the evaporator. The vapor deriving from the evaporator is fed to the degasser which contains some perforated plates which serve to facilitate both the separation and the contact of the two phases, the liquid one and the vapor one. The vapor phase that leaves the degasser is partially condensed in a reflux type condenser, which operates at a temperature of 20-250° C., preferably 40-150° C., even more preferably at 60-130° C.; optionally, further condensation can be carried out to recover also by-products that may be formed during the preceding reactions.

The vapors leaving the reflux condenser are condensed, in another condenser, at a temperature between 2-50° C., preferably 10-30° C., more preferably 20° C.

The liquid that collects at the outlet of the last condenser is a mixture of solvent and water. The solvent, after the separation of the water, can be recycled, while the mixture that comes out from the bottom of the evaporator can be sent to the dehydration step.

Dehydration takes place continuously in a reactor, called dehydrator, preferably of the tubular type, equipped with a heating system and a condensing system formed by a condenser which condenses most of the water that is produced and which sends the condensates to a phase separator. In the phase separator any traces of organic are separated and reintroduced into the dehydrator, while the water can be partly recycled to a hydrogenation section which can be arranged upstream and the excess sent for treatment. In a preferred embodiment, the mixture is fed continuously laterally into the reactor while the steam exits from the reactor head and the reaction product exits from the bottom. Said reactor can optionally contain, in the upper part, fillings such as e.g. rings, plates, septa, such as to favor the release of water vapor only. In another embodiment, the reaction mixture can also be fed continuously from the bottom and the reaction product withdrawn laterally from the reactor while the water vapor exits from the reactor head.

The reaction is carried out in the presence of a heterogeneous acid catalyst, preferably γ-alumina, with a WHSV (Weight Hourly Space Velocity, relative to the entire reagent mixture) between 1 and 50 h⁻¹, preferably between 3 and 10 h⁻¹. The dehydration is carried out hot, preferably between 90 and 270° C., more preferably between 130 and 230° C., even more preferably between 150 and 200° C.

The pressure at which the reaction is carried out is comprised between 0.08 and 5 BarA, preferably between 0.5 and 3 BarA, more preferably between 1 and 2 BarA. A stream formed by the dehydration products, any solvent and unreacted amine, and the by-products coming from the previous steps comes out from the bottom of the reactor; in the case in which the compound of formula (IV) is N-(3-aminopropyl)-ε-caprolactam, the main product is typically DBU (1,8-Diazabicyclo[5.4.0]undec-7-ene).

Said stream of products is then sent to a distillation section for the purification of the compound (V), such as for example the DBU. After distillation, the purity of said compound is typically between 95 and 98%.

Purity of said compound is determined by gas chromatographic analysis (GC-MS).

Optionally, said compound, after distillation, can be subjected to further purifications such as liquid-liquid extractions. These operations can be carried out with the techniques known to the skilled in the art.

### EXAMPLES

Unless otherwise indicated, the following examples refer to the following abbreviations and materials:
AN: acrylonitrile (CAS 107-13-1, purity≥99%, Sigma-Aldrich)
CPLT: ε-caprolactam (CAS 105-60-2, purity 99%, Sigma-Aldrich)
NaOH: sodium hydroxide (CAS 1310-73-2, purity≥98%, Sigma-Aldrich)
iPrOH: iso-propanol (CAS 67-63-0, purity 99.5%, Acros Organics)
Xylene: mixture of xylene isomers (CAS1330-20-7, purity ≥98.5%, Sigma-Aldrich)
Tetrahydrofuran (THF - CAS 109-99-9, purity ≥99.0%, Sigma-Aldrich)
CTZ1: commercial catalyst HTC Co 2000 RP 1.2 mm (Co≈15% supported on alumina) Johnson-Matthey (data from the U.S. Pat. No. 8,293,676 B2 Table 3 columns 21-22 Example J)
CTZ2: commercial catalyst HTC Ni 500 Johnson-Matthey (in the form of 1.2 mm trilobular shaped extruded material containing 21% nickel as nickel oxide, on a porous transition alumina support, data from international patent application (PCT) WO 2010/018405 example 1 on page 6)
H2: hydrogen (Sapio Title 5.5)
H2O: ultra pure water (MilliQ millipore system)
Alumina (SASOL SPHERES 1.0/160)

### Gas-Mass Analysis

The gas-mass analysis for the determination of reagents and reaction products is carried out with a GC HP6890 chromatograph, equipped with a split/splitless injector and interfaced with a MS HP 5973 mass spectrometer acting as a detector. The chromatograph has an HP-1MS UI capillary column (100% polydimethylsiloxane, Agilent J&W), fused silica WCOT, 30 m long, 0.25 mm ID, 0.25 μm film thickness. The instrumental parameters are as follows:
injection volume 20 μl
Carrier gas helium at 0.8 ml/min (constant flow mode)
split ratio 250:1
Injector temperature 300° C.
programmed oven temperature from 40° C. to 320° C. at 10° C./min (28 min) plus hold time of 10 min at 320° C. (total run time=38 min). In the absence of the availability on the market of some pure products (c-caprolactam amine), the quantification was performed with the method of comparing the relative areas to the various chromatographic peaks (thus accepting the approximation that they all have the same chromatographic response). However, a ¹H and ¹³C NMR quantitative analysis was also carried out on the same samples; the results obtained were comparable to those illustrated with the chromatographic technique.

### NMR Analysis

The analyzes on the samples provided were carried out using the Bruker Avance 400 MHz spectrometer, at a temperature of 300 K, dissolving about 50-70 mg of sample in deuterated chloroform. Spectra were recorded with the following instrumental parameters:

| | PROBE: 5 mm PABBO BB/¹⁹F—¹H/D Z-GRAD | | | |
|---|---|---|---|---|
| | ¹H - 400 MHz | | ¹³C - 100 MHz | |
| Method | zg30 | | zgpg30 | |
| No. scans | 64 | | 512 | |
| No. data point | 32k | | 32k | |
| p1 (μs) | 26.00 | | 17.00 | |
| d1 (s) | 7.00 | s | 8.00 | s |
| Spooky window | 15 | ppm | 240 | ppm |
| O1P | 4.0 | ppm | 100.0 | ppm |
| PL1 | −0.85 | dB | −0.85 | dB |

| | |
|---|---|
| Solvent | CDCl₃ 99.9 atom % D + ~0.1% TMS |

### Preparation 1: Reaction Between ε-Caprolactam and Acrylonitrile in Iso-Propanol

250.4 g of ε-caprolactam and 127.2 g of iso-propanol were placed in a 1 liter flask equipped with a nitrogen inlet, stirrer, reflux refrigerant, thermocouple and dropping funnel. The suspension under stirring was heated to 45-50° C. under a light flow of nitrogen by using an oil bath; when completely dissolved, 0.36 g of NaOH were added and the temperature was brought to 70° C. Once the sodium hydroxide had been solubilized, acrylonitrile (129.0 g) began to drop, taking care to keep the temperature between 70-80° C.; the reaction was exothermic. At the end of the addition of the acrylonitrile the temperature was brought to 80° C. and it was allowed to react for 1.5 h; a progressive browning of the solution was noted as the addition reaction progressed. GC-MS analysis revealed a conversion of caprolactam of 96.1%, a selectivity of 90.5% and therefore a yield in the product of 87.0%. The basic crude solution was subjected to hydrogenation as described nelow in preparation 4.

### Preparation 2: Reaction Between ε-Caprolactam and Acrylonitrile in Xylene

The same reaction described in preparation 1 was carried out by replacing iso-propanol with xylene and making it react for 2.25 h at the end of the addition of the acrylonitrile (70° C.). GC-MS analysis revealed a conversion of caprolactam of 98.6%, a selectivity of 98.3% and therefore a yield in the product of 96.9%. The basic crude solution was subjected to hydrogenation as described below in preparations 5 and 6.

### Preparation 3: Reaction Between ε-Caprolactam and Acrylonitrile in Tetrahy Drofuran

The same reaction described in preparation 1 was carried out by replacing iso-propanol with tetrahydrofuran and making it react for 2.25 h at the end of the addition of the acrylonitrile (70° C.). GC-MS analysis revealed a conversion of caprolactam of 98.2%, a selectivity of 98.5% and therefore a yield in the product of 96.7%. The basic crude solution was subjected to hydrogenation as described below in preparations 7 and 8.

### Preparation 4: Hydrogenation of the Crude Solution in Iso-Propanol With Co Catalyst

In a 250 ml autoclave equipped with a mechanical turbine stirrer, heating mantle, basket for the catalyst, inlet for gases and liquid streams, 30 g of CTZ1 catalyst were introduced, at room temperature, into the dedicated catalyst holder, and activated in a hydrogen atmosphere.

Activation of the catalyst was carried out by first subjecting it to flushing with nitrogen at atmospheric pressure, after which the reactor was heated up to 150° C. with a temperature ramp of 25-50° C./h, and, once reached such temperature, there has been proceeded with the supply of hydrogen at a flow rate of 30 ml/min, thus raising the temperature up to 180° C.

At this point, the hydrogen flow rate was increased, progressively reducing the nitrogen flow rate until the gas flushing was completely hydrogen (flow rate 200 ml/min). In these conditions of temperature and flow rate, the activation continued for 18 hours, then the nitrogen current was restored (and that of hydrogen at the same time reduced) in order to keep the catalyst in an inert atmosphere, progressively cooling the system up to room temperature.

4.5 g of H 2 O (approx. 3% with respect to the total) were added to 143.7 g of solution obtained from preparation 1 and then loaded into the reactor; thereafter, the lines were washed by introducing a further 19.0 g of iso-propanol. The reactor pressure was brought to 21 barA, by activating the stirrer motor (750 rpm) and turning on the heating and setting an internal temperature of 130° C. In the meantime, there has been continued to pressurize with hydrogen, thus reaching the temperature of 130° C. at the desired pressure of 41 barA. It was hydrogenated at this pressure as long as there was a flow of hydrogen of about 0.2-0.3 L/h in line to the reactor. The indication of the volume of hydrogen introduced into the reactor through the meter was also used and the same was compared with the stoichiometric quantity calculated on the basis of the quantity of nitrile introduced. Finally the product was cooled and discharged.

GC-MS analysis revealed a conversion of the nitrile product equal to 97.9%, a selectivity of 98.9% and therefore a yield in the products N-(3-aminopropyl)-ε-caprolactam and DBU (1.8-Diazabicycle[5.4.0]undec-7-ene) of 96.8%.

### Preparation 5: Hydrogenation of the Crude Solution in Xylene With Co Catalyst

The same reaction described in preparation 4 was carried out using the mixture coming from preparation 2 and containing xylene instead of iso-propanol. The sample obtained was subjected to GC-MS analysis. The results are reported in table 2.

### Preparation 6: Hydrogenation of the Crude Solution in Xylene With Ni Catalyst

The same reaction described in preparation 5 was carried out using the commercial catalyst CTZ2, instead of the catalyst CTZ1 (the activation mode is similar to that already described above). The sample obtained was subjected to GC-MS analysis. The results are reported in table 2.

### Preparation 7: Hydrogenation of the Crude Solution in THF With Co Catalyst

The same reaction described in preparation 4 was carried out using the mixture coming from preparation 3 and containing THF instead of iso-propanol. The sample obtained was subjected to GC-MS analysis. The results are reported in table 2.

### Preparation 8: Hydrogenation of the Crude Solution in THF With Ni Catalyst

The same reaction described in preparation 7 was carried out using the commercial catalyst CTZ2, instead of the catalyst CTZ1. The sample obtained was subjected to GC-MS analysis. The results are reported in table 2.

### Preparation 9: Reaction Between Caprolactam and Acrylonitrile in the Absence of Solvent

The same reaction described in preparations 1-3 was also carried out in the absence of solvent.

123.4 g of c-caprolactam were placed in a 500 ml flask equipped with a nitrogen inlet, stirrer, reflux cooler, thermocouple and dropping funnel. The solid was heated to 70-75° C. under a light flow of nitrogen by using an oil bath (external temperature control); when completely melted, 0.1230 g of NaOH were added and the temperature was brought to 70° C. (internal temperature control). Once the sodium hydroxide was solubilized, acrylonitrile (67.4 g) began to drop, taking care to keep the temperature between 70-80° C.; the reaction was exothermic. At the end of the addition of acrylonitrile, the temperature was maintained at 70° C. and it was allowed to react for 2 h; a progressive browning of the solution was noted as the addition reaction progressed.

### Example 1

### Dehydration of the Crude Solution in Xylene

The solution coming from preparation 5 (138.3 g) was introduced into a flask (containing a few glass balls), connected to a Dean-Stark apparatus equipped with bubble refrigerant. One gram of SASOL alumina SPHERES 1.0/160 previously activated in an oven at 150° C. for 8 hours was then added. The flask was heated up to 170° C.; the water that formed from the reaction was separated while the solvent was recovered. After about 4 h there was no more water formation; the flask was then cooled and the contents were subjected to GC-MS analysis. The analysis calculated a conversion of N-(3-aminopropyl)-ε-caprolactam equal to 94.7%, a selectivity of 99.5% and therefore a yield in DBU of 94.2%.

### Example 2: Dehydration of the Amine with Heterogeneous Acid Catalyst Without Solvent

The solvent from the preparation 5 was removed with a rotavapor (T=60° C.; P=30 mbar) thus obtaining 113.9 g of a mixture of N-(3-aminopropyl)-ε-caprolactam and DBU; this solution was introduced into a flask (containing a few glass balls), connected to a Liebig condenser for the removal of the reaction water. One gram of SASOL alumina SPHERES 1.0/160 previously activated in an oven at 150° C. for 8 hours was then added. The dehydration was carried out in a light nitrogen flow to facilitate the elimination of the water. The flask was then heated up to 170-180° C. for about 5 h (time at which no more condensate formation was noted); the flask was then cooled and the contents were subjected to GC-MS analysis. The analysis calculated a conversion of N-(3-aminopropyl)-ε-caprolactam equal to 93.6%, a selectivity of 83.1% and therefore a yield in DBU of 77.8%.

Tables 1, 2 and 3 show the summary data of the previous examples.

Finally, it is understood that further modifications and variants not specifically mentioned in the text may be made to the process as described and illustrated herein, which however are to be considered as obvious variants of the present disclosure within the scope of the annexed claims.

### Calculation of Conversions, Selectivity and Yields (GC-MS)

$$conv \% = \frac{mol initial reagent - mol residual reagent}{mol initial reagent} ⋆ 100$$

$$sel \% = \frac{mol obtained product}{mol reacted reagent} ⋆ 100 yield \% = \frac{conv \% ⋆ sel \%}{100}$$

### Where:

mol=number of moles

TABLE 1
addition reactions

| Preparation | 1 | 2 | 3 | 9 |
|---|---|---|---|---|
| CPLT (g) | 250.4 | 123.3 | 147.4 | 123.4 |
| AN (g) | 129.0 | 67.4 | 80.1 | 67.4 |
| NaOH (g) | 0.3603 | 0.1841 | 0.216 | 0.1230 |
| Solvent (≈25% w/w) | IPrOH | Xylene | THF | — |
| Temperature (° C.) | 80 | 70 | 70 | 70 |
| Time (1) (h) | 1.5 | 2.25 | 2.25 | 2 |
| Pressure (barg) | 0 | 0 | 0 | 0 |
| CPLT Conv. (%) | 96.1 | 98.6 | 98.2 | 95.4 |
| Nitrile selec. (%) | 90.5 | 98.3 | 98.5 | 98.9 |
| Nitrile yield (%) | 87.0 | 96.9 | 96.7 | 94.4 |

(1) Reaction time at the end of the dosage of the AN (the dosage time for all examples is 1 h)

TABLE 2
reduction reactions

| Preparation | 4 | 5 | 6 | 7 | 8 |
|---|---|---|---|---|---|
| Nitrile compound | From | From | From | From | From |
| in solution (ref.) | preparation 1 | preparation 2 | preparation 2 | preparation 3 | preparation 3 |
| Nitrile Compound | 143.7 | 143.9 | 143.0 | 144.0 | 143.9 |
| Mass (g) | | | | | |
| Water (% w/w | 2.69 | 2.67 | 2.84 | 2.73 | 2.67 |
| with respect to tot) | | | | | |
| Solvent (≈25% w/w) | IPrOH | Xylene | Xylene | THF | THF |
| CTZ (metal | 30 g CTZ1 | 30 g CTZ1 | 30 g CTZ2 | 30 g CTZ1 | 30 g CTZ2 |
| supported on | (Co) | (Co) | (Ni) | (Co) | (Ni) |
| Al₂O₃, g) | | | | | |
| Agitation speed | 750 | 750 | 750 | 750 | 750 |
| (rpm) | | | | | |
| T (° C.) | 130 | 130 | 160 | 130 | 150 |
| P H₂ (Barg) | 40 | 40 | 40 | 40 | 40 |
| Conv. Nitrile (%) | 97.9 | 96.1 | 96.9 | 94.1 | 96.3 |
| Select. (2) (%) | 98.9 | 99.3 | 78.5 | 99.2 | 79.7 |
| Yield (2) (%) | 96.8 | 95.4 | 76.1 | 93.3 | 76.8 |

(2) Yield and selectivity towards all desired products (primary amine plus DBU)

TABLE 3
dehydration reactions

| Example | 2 |
|---|---|
| Amine compound in | From |
| solution (ref.) | preparation 5 |
| Primary amine/DBU | 113.9 |
| mixture mass (g) without | |
| solvent | |
| CTZ mass (Al₂O₃, SASOL | 1 g |
| Spheres 1.0/160, g) | |
| T (° C.) | 170-180 |
| P (Barg) | 0 |
| Primary amine Conv. (%) | 93.6 |
| DBU Select. (%) | 83.1 |
| DBU yield (%) | 77.8 |

Considering in succession the results of preparations 2 and 5 and of example 2 it was possible to calculate the overall yield of the synthesis:

| Preparation 2 | Preparation 5 | Example 2 | |
|---|---|---|---|
| yield (%) | yield (%) | yield (%) | Total yield (%) |
| 96.9 | 95.4 | 77.8 | 71.9 |

1. A process for preparing amidines or derivatives thereof of formula (V) 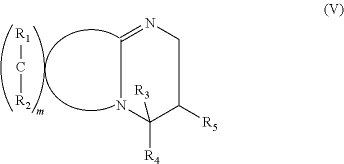
starting from a compound of formula (IV): 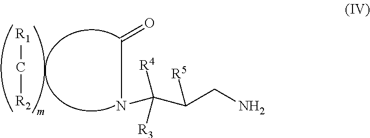
wherein:
R1 is H or an aliphatic hydrocarbon group, optionally substituted, having from 1 to 5, preferably from 1 to 2 carbon atoms, and is more preferably H;
R2 is H or an aliphatic hydrocarbon group, optionally substituted, having from 1 to 5, preferably from 1 to 2 carbon atoms, and is more preferably H;
R3 is H or an aliphatic hydrocarbon group, optionally substituted, having from 1 to 5, preferably from 1 to 2 carbon atoms, and is more preferably H;
R4 is H or an aliphatic hydrocarbon group, optionally substituted, having from 1 to 5, preferably from 1 to 2 carbon atoms, and is more preferably H;
R5 is H or an aliphatic hydrocarbon group, optionally substituted, having from 1 to 5, preferably from 1 to 2 carbon atoms, and is more preferably H;
m is an integer from 3 to 7, more preferably 3 to 6,
wherein said process comprises the step of subjecting said compound of formula (IV) to dehydration in the presence of an acid catalyst,
wherein the acid catalyst is selected from heterogeneous acid catalysts selected from Lewis acids or Lewis acids with Bronsted acid components, or heterogeneous acid catalysts based on acid resins.

2. The process according to claim 1, wherein the catalyst is selected from aluminum oxide (γ-Al₂O₃) and silico-aluminas (SiO₂—Al₂O₃).

3. The process according to claim 1, wherein the catalyst is selected from earth acids selected from lanthanum oxide and zirconium oxide.

4. The process according to claim 1, wherein the catalyst is selected from sulfonated resins or ion exchange resins.

5. The process according to claim 1, wherein the catalyst is supported/bound on an inert carrier.

6. The process according to claim 1, wherein the dehydration reaction is carried out in the absence of solvent, at a temperature between 90 and 270° C., or between 130 and 230° C., or between 150 and 200° C., continuously removing the water produced during dehydration.

7. The process according to claim 1, wherein the dehydration reaction is carried out in the presence of a solvent selected from xylenes and ethylbenzene, at a temperature between 90 and 270° C., continuously removing the water produced during dehydration.

8. The process according to claim 1, wherein the dehydration reaction is carried out continuously in a tubular-type reactor, equipped with a heating system and a condensing system formed by a condenser which condenses most of the water which is produced and which sends the condensates to a phase separator, and comprising the separation in the phase separator of any traces of organic products and their reintroduction into the dehydration reactor.

9. The process according to claim 1, wherein the dehydration reaction is carried out continuously in a tubular-type reactor with a WHSV (Weight Hourly Space Velocity, relative to the entire reagent mixture) between 1 and 50 h⁻¹.

10. The process according to claim 9, wherein the dehydration reaction is carried out at a pressure comprised between 0.08 and 5 BarA.

11. The process according to claim 1, wherein the compound of formula (V) is 1,8-Diazabicyclo[5.4.0]undec-7-ene (DBU).
